(19) United States
(12) Patent Application Publication
(10) Pub. No.: US 2007/0239031 A1
(43) Pub. Date: Oct. 11, 2007

(54) Systems and methods for performing simultaneous tomography and spectroscopy

(76) Inventors: Kye-Sung Lee, Orlando, FL (US); Jannick Rolland, Chuluota, FL (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/404,322

(22) Filed: Apr. 14, 2006

Related U.S. Application Data
(60) Provisional application No. 60/773,486, filed on Feb. 15, 2006.

(51) Int. Cl. A61B 6/00

(52) U.S. Cl. 600/473

(57) ABSTRACT

Systems and method for performing simultaneous optical coherence tomography and spectroscopy. In one embodiment, a system includes a light source that emits light to be delivered to a material under evaluation, and a receiver that collects both light that is backscattered by features of the material and fluorescent light that is emitted by features of the material. In one embodiment, a method includes simultaneously collecting near-infrared light backscattered by a material under evaluation and fluorescent light emitted by the material under evaluation using a single light detector.

### CROSS-REFERENCE TO RELATED APPLICATION

This application claims priority to copending U.S. provisional application Ser. No. 60/773,486, entitled, “Optical Apparatuses and Methods,” filed Feb. 15, 2006, which is hereby incorporated by reference in its entirety.

### BACKGROUND

Cancer is a major public health problem in the United States and other developed countries. According to the American Cancer Society (ACS), one in four deaths in the United States is due to cancer, of which skin cancer is the most common form. One in five Americans will contract skin cancer in the course of a lifetime and, on average, one person dies every hour from skin cancer, primarily melanoma, the most deadly form of skin cancer.

Although melanoma can quickly spread to other body parts, it is curable if detected early and properly treated. For most present-day medical practitioners, the final cancer or pre-cancer diagnosis is based on excisional (surgical) biopsy. To date, excisional biopsy has been the only certain method to determine if a growth is cancerous. While excisional biopsy is the standard method for cancer detection, many biopsies are done on a hit-or-miss basis because only small pieces of tissue are excised at random and dissected to check for cancerous cells. Moreover, excisional biopsy imposes problems, like the risk of cancer cell spreading, infection, and hemorrhage.

Due to the invasiveness of excisional biopsy, there is a present desire for a non-invasive, early-stage method for detecting cancer or pre-cancer. Photonics solutions have carried justified hopes in providing such a non-invasive method. One such photonics solution is optical coherence tomography (OCT). OCT can be used to capture high-resolution, cross-sectional images of tissues, such as the skin, to facilitate diagnosis of cancer and pre-cancer. Another photonics solution is fluorescence spectroscopy. Fluorescence spectroscopy can be used to capture cross-sectional images of fluorescent light emitted from features within tissue that may be indicative of cancer or pre-cancer.

Recently it has been proposed to use OCT in conjunction with fluorescence spectroscopy to diagnose cancer or pre-cancer. The desirable optical sectioning of OCT combined with the information provided by fluorescence spectroscopy enables imaging of microscopic structures in tissues at depths well beyond the reach of conventional confocal microscopes and simultaneously provides valuable chemical composition information about the tissue.

Current systems for simultaneously performing OCT and fluorescense spectroscopy require separate light detectors for the OCT and the spectroscopy information obtained from the tissue under evaluation. Given the expense and complexity of such systems, it would be desirable to have a system and method for simultaneously performing OCT and fluorescence spectroscopy that uses a single light detector that collects both the OCT/OCM and the spectroscopy information.

### SUMMARY

Disclosed are systems and method for performing simultaneous optical coherence tomography and spectroscopy. In one embodiment, a system includes a light source that emits light to be delivered to a material under evaluation, and a receiver that collects both light that is backscattered by features of the material and fluorescent light that is emitted by features of the material.

In one embodiment, a method includes simultaneously collecting near-infrared light backscattered by a material under evaluation and fluorescent light emitted by the material under evaluation using a single light detector.

### BRIEF DESCRIPTION OF THE FIGURES

The components in the figures are not necessarily to scale, emphasis instead being placed upon clearly illustrating the principles of the present disclosure. In the figures, like reference numerals designate corresponding parts throughout the several views.

FIG. 1 is a schematic view of a first embodiment of an imaging system that can perform simultaneous tomography and spectroscopy.

FIG. 2 is a schematic view that depicts a receiver shown in FIG. 1 simultaneously collecting backscattered light and fluorescent light with a single light detector.

FIG. 3 is a schematic view of a second embodiment of an imaging system that can perform simultaneous tomography and spectroscopy.

FIG. 4 is a schematic view of a third embodiment of an imaging system that can perform simultaneous tomography and spectroscopy.

FIG. 5 is a schematic view of a fourth embodiment of an imaging system that can perform simultaneous tomography and spectroscopy.

FIG. 6 is a schematic view of a fifth embodiment of an imaging system that can perform simultaneous tomography and spectroscopy.

FIG. 7 is a schematic view of a sixth embodiment of an imaging system that can perform simultaneous tomography and spectroscopy.

FIG. 8 is a schematic view that depicts imaging of various portions of a material under evaluation.

FIG. 9 is a flow diagram that illustrates an embodiment of a method for performing simultaneous tomography and spectroscopy.

### DETAILED DESCRIPTION

As described above, there is a current desire for photonics solutions that may be used to aid in the detection and diagnosis of cancer or pre-cancer. More particularly, desired are systems that can simultaneously perform optical coherence tomography (OCT) and fluorescence spectroscopy to aid in the detection and diagnosis of cancer or pre-cancer. Unfortunately, current systems require separate light detectors for the OCT and the spectroscopy information obtained from the tissue under evaluation.

As described in the following, disclosed are systems and methods for performing simultaneous tomography and spectroscopy in which a single receiver or detector is used to collect the information used in both the tomography and spectroscopy. In some embodiments, Fourier-domain OCT is simultaneously performed along with two-photon fluorescence spectroscopy. In such a case, high-resolution morphological (i.e., structural) information and biochemical information about the tissue under evaluation can be obtained. Moreover, OCT images and fluorescence spectroscopy images of discrete portions of the tissue can be generated that can be compared or superimposed on top of each other for visual inspection and computer analysis.

In the following, described are various embodiments of systems and methods for performing simultaneous tomography and spectroscopy. Although particular embodiments are described, the disclosed systems and methods are not limited in their application to those particular embodiments. Instead, the described embodiments are mere example implementations of the disclosed systems and methods. Furthermore, although the systems and methods are described as being particularly suitable for use in the detection and diagnosis of cancer and pre-cancer of animal tissue, it is to be understood that the methods and systems are not limited to that application and can be used to image and evaluate tissue, or non-biological materials, for other purposes.

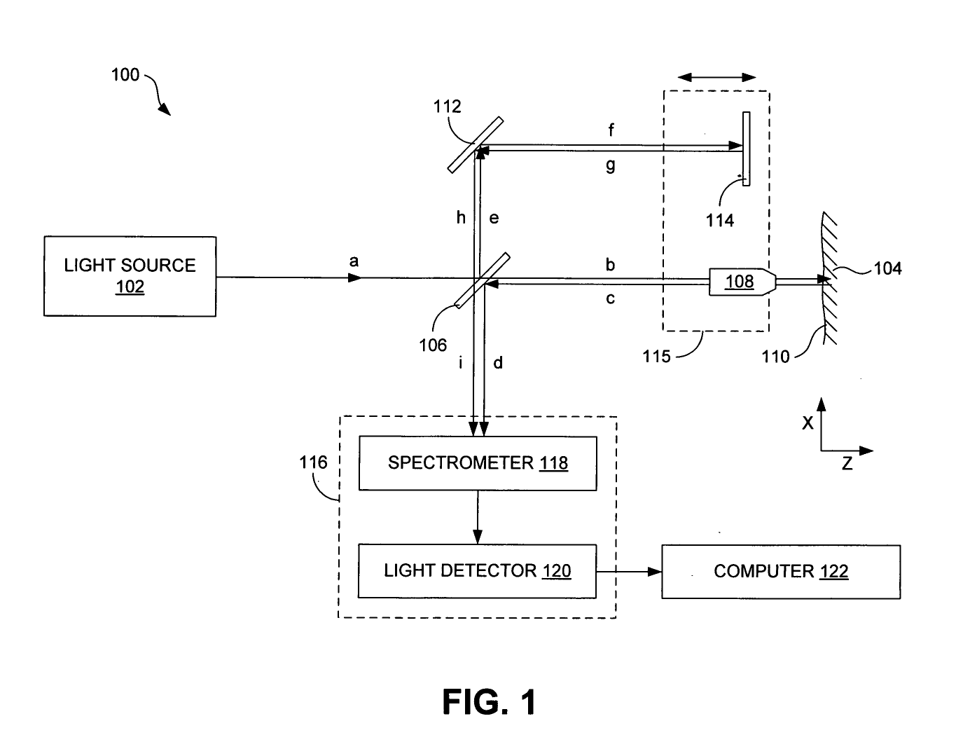

FIG. 1 illustrates a first embodiment of a system 100 for simultaneously performing OCT and fluorescence spectroscopy. It is noted that, as used herein, OCT is intended to include optical coherence microscopy (OCM), which is considered a specific variant of OCT. As indicated in FIG. 1, the system 100 comprises a light source 102 that is used to illuminate material 104 under evaluation, such as animal (e.g., human tissue). More particularly, the light source 102 emits high-intensity, low-coherence, near-infrared (NIR) light toward the material 104. By way of example, the light source 102 comprises a pulsed infrared laser, such as a mode-locked, titanium-doped sapphire (Ti:Sa) femto-laser. The light source 102 has a central wavelength in the range of approximately 700 nanometers (nm) to 900 nm, for example 800 nm, and a spectral bandwidth of approximately 120 nm. As described in the following, the light source 102 can be tunable to emit high-power pulses that enable two-photon excitation of features contained in the material 104. For example, the light source may emit pulses having a peak power of approximately a few hundred kilowatts (kW).

Positioned between the light source 102 and the material 104 under evaluation is a beam splitter 106 and an objective 108. The beam splitter 106 is configured to both reflect and transmit light in the visible and NIR spectra and, for example, comprises a 50/50 beam splitter. Therefore, the light emitted by the light source 102 can pass through the beam splitter 106 and be focused by the objective 108 on a desired location of the material 104, for example at a point below the surface 110 of the material. By way of example, the objective 108 has a numerical aperture of 0.3, which yields a transverse resolution of approximately 1.6 microns (μm) and a depth of focus of approximately 20 μm.

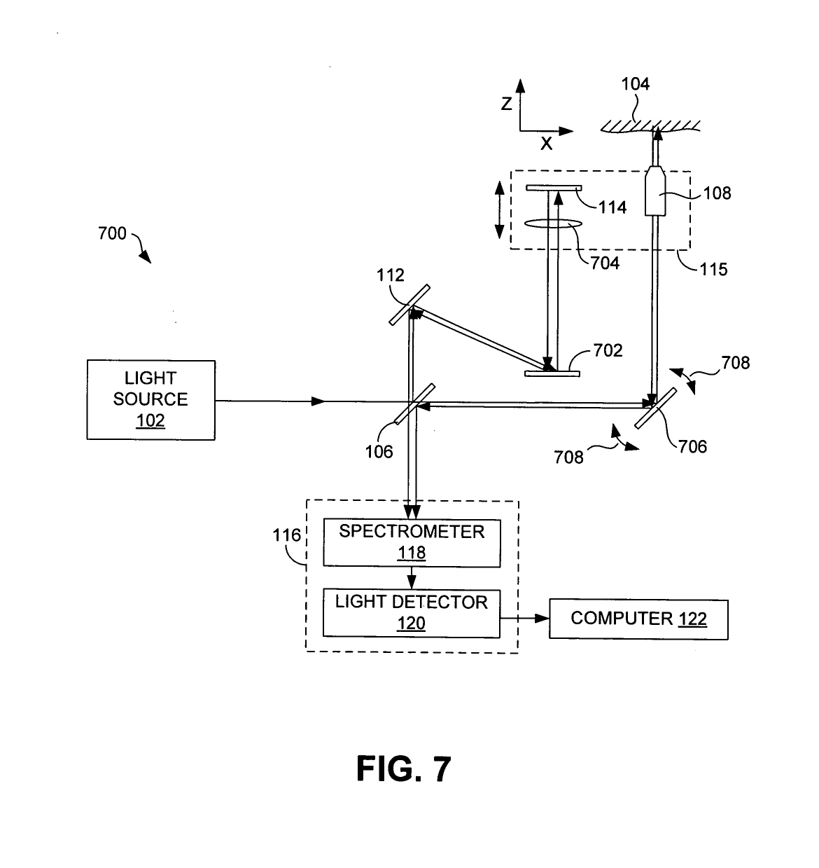

In addition, the system 100 comprises mirrors 112 and 114, which form part of a reference path for the light emitted by the source 102. As shown in FIG. 1, the mirror 114 (“reference” mirror) can be mounted to a structure 115, such as a microscope stage, to which the objective 108 is mounted. With such an arrangement, the objective 108 and the mirror 114 can be displaced in a depth or “z” direction. It is noted that although specific orthogonal directions have been identified, they are identified by way of example and may be alternatively defined. For example, the “x” direction shown in FIG. 1 may be defined to comprise the “y” direction. The transverse (x and/or y) scanning can be directly moving the material 104 laterally using a stepper motor or, as described in relation to FIG. 7, by deviating the light applied to the material.

Further comprised by the system 100 is a receiver 116 that collects light information that is backscattered (OCT) and emitted (fluorescence spectroscopy) by the material 104 under evaluation. As indicated in FIG. 1, the receiver 116 comprises a spectrometer 118 that spreads the light received from the material 104 and a light detector 120, such as a charge-coupled device (CCD), photodiode array, or photomultiplier array, that detects the intensity of the spread light.

In communication with the receiver 116 is a computer 122 that can be used to manipulate intensity data from the light detector 120. Such manipulation can comprise the generation of images and/or qualitative analysis of the data.

As described above, the system 100 can be used to perform Fourier-domain OCT. To that end, NIR light is emitted by the light source 102 along path a. A portion of that light is transmitted by the beam splitter 106 toward the objective 108 along path b. The objective 108 focuses the light at a desired location within the material 104 under evaluation. Some of that light is then backscattered by features contained within the material 104 and travels back through the objective 108 toward the beam splitter 106 along path c. A portion of that light is then reflected by the beam splitter 106 along path d to the receiver 116.

Simultaneous to the above, a portion of the light emitted by the light source 102 is reflected by the beam splitter 106 along path e. That light is reflected by the mirror 112 and travels along path f toward the mirror 114. The mirror 114 reflects the light back toward the mirror 112 along path g. The mirror 112 then reflects that light toward the beam splitter 106 along path h. A portion of that light travels through the beam splitter 106 toward the receiver 116 along path i.

With the above-described light propagation, the receiver 116 receives both a sample signal from the signal path defined by paths b and c, and a reference signal from the reference path defined by paths e, f, g, and h. Because the reference path is configured so as to have an optical length that is substantially equal to that of the sample path, interference will occur at the receiver 116 such that a spectrally measured interferogram is generated that contains information about the structural features of material 104.

In addition to performing Fourier-domain OCT, the system 100 simultaneously performs two-photon fluorescence spectroscopy. In that regard, light emitted by the light source 102 travels along paths a and b in the manner described above. With appropriate tuning of the light source 102 and focusing of the objective, the light is highly concentrated on features of the material 104 under evaluation so as to cause two-photon excitation that results in emission of visible, fluorescent light from those features. When that occurs, the fluorescent light has a wavelength that is approximately half the wavelength of the NIR light emitted by the light source 102. Therefore, if the light source 102 emits light having a central wavelength of approximately 800 nm, fluorescent light having a wavelength of approximately 400 nm is emitted by the material features. Although such fluorescence may occur naturally, a suitable fluorescent dye can be applied to the material 104 to enable or increase fluorescence.

The emitted fluorescent light travels along path c to the beam splitter 106, which reflects the light toward the receiver 116 along path d. Therefore, the receiver 116 receives both the NIR light that is backscattered by the material and the fluorescent light that is emitted by the material.

Significantly, the use of Fourier-domain OCT, as opposed to other OCT methodologies such as time-domain OCT, enables the use of a single receiver 116, and therefore a single light detector 120, in capturing OCT and spectroscopy data. Specifically, because Fourier-domain OCT is performed by collecting spectra, a single receiver 116 and a single light detector 120 can be used to collect the spectra associated with both the OCT and the spectroscopy. Because the OCT signals are NIR spectra and the fluorescence spectroscopy signals are visible spectra, no spectral overlap occurs as between the OCT and the spectroscopy signals.

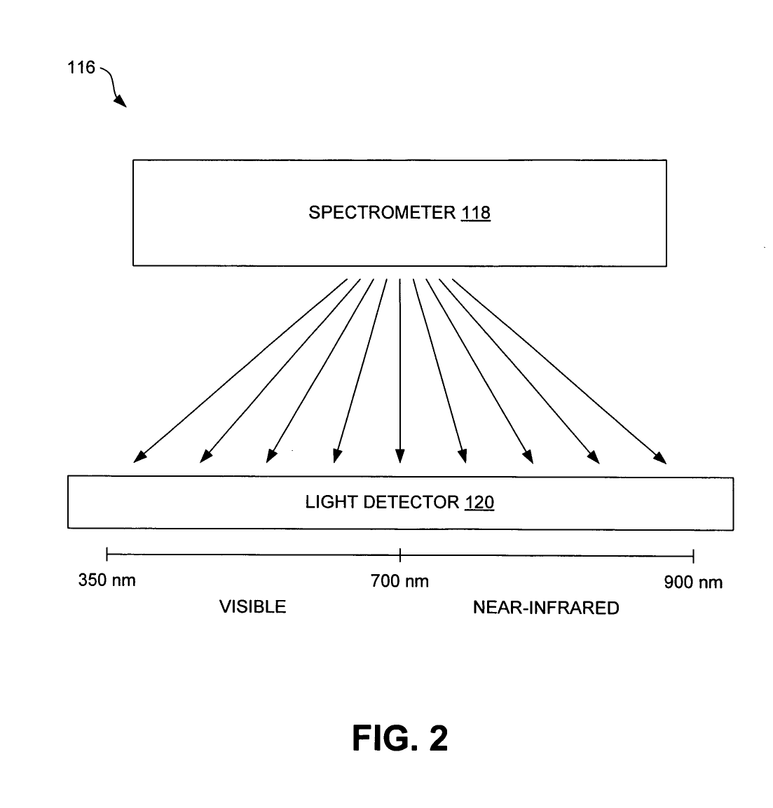

FIG. 2 schematically depicts the spreading of spectra by the spectrometer 118 and the collection of that spectra with the light detector 120. As indicated in that figure, the spectrometer 118 spreads the spectra such that different wavelengths of light impinge upon the light detector 120 at different locations. For example, in the embodiment of FIG. 2, light impinges upon the light detector 120 from lowest wavelength to highest wavelength from one end of the detector to the other. Therefore, the backscattered NIR light used in the OCT (e.g., at about 800 nm) will impinge upon the detector 120 in the NIR portion of the detector, while the fluorescent light used in the fluorescence spectroscopy (e.g., at about 400 nm) will impinge upon the detector in the visible portion of the detector. Given that the computer 122 (FIG. 1) is provided with correlation information that correlates the various pixel positions of the detector 120 with light wavelengths, the appropriate OCT and spectroscopy manipulation can be performed by the computer.

As stated above, the manipulation performed by the computer 122 can comprise the generation of OCT and fluorescence spectroscopy images that can be, for example, displayed for a medical practitioner. Given that those images are simultaneously-captured images of the discrete portions of the material, they can be displayed in association with each other for easy comparison, or can be superimposed on top of each other. In addition, the computer 122 can analyze the image data according to one or more algorithms to aid in the detection or diagnosis of a phenomenon, such as disease. For example, the computer 122 can identify the boundaries of layers of skin and calculate layer thicknesses from the structural data that results from the OCT. In addition, the computer 122 can identify features within the spectroscopy data that are considered abnormal as determined by the observed wavelengths and/or intensity of the fluorescent light. Such analyses may be facilitated by a calibration process in which the characteristics of “normal” tissue are recorded for purposes of comparison (e.g., as a control).

The use of two-photon fluorescence spectroscopy is desirable for several reasons. First, two-photon fluorescence spectroscopy enables greater imaging depth. Second, two-photon fluorescence spectroscopy enables the use of a single, NIR light source. Generally speaking, a fluorescent light source could be used to illuminate features of the material under evaluation. However, two-photon excited fluorescence, which occurs when two IR photons simultaneously collide with a feature, excites the feature to a state virtually identical to that caused by a single visible photon of about half the wavelength such that the feature emits a visible photon. Therefore, instead of illuminating the material with an NIR source for OCT and a separate fluorescent source for fluorescence spectroscopy, an NIR source alone can be used in the system. In addition to reducing the complexity of the system, avoiding the use of a separate fluorescent source also reduces noise that would occur in the form of light signals received from the source in the fluorescent signal. Third, and perhaps most significant, the use of two-photon fluorescence spectroscopy enables the collection of fluorescent light from discrete points of the material under evaluation rather than a general, undefined region because two-photon absorption only occurs at points of high light intensity (i.e., the focus point). Therefore, the fluorescent light is spatially resolved and coincident with the backscattered NIR light so that the OCT and spectroscopy images are automatically registered with each other, thereby enabling direct comparison or superimposition.

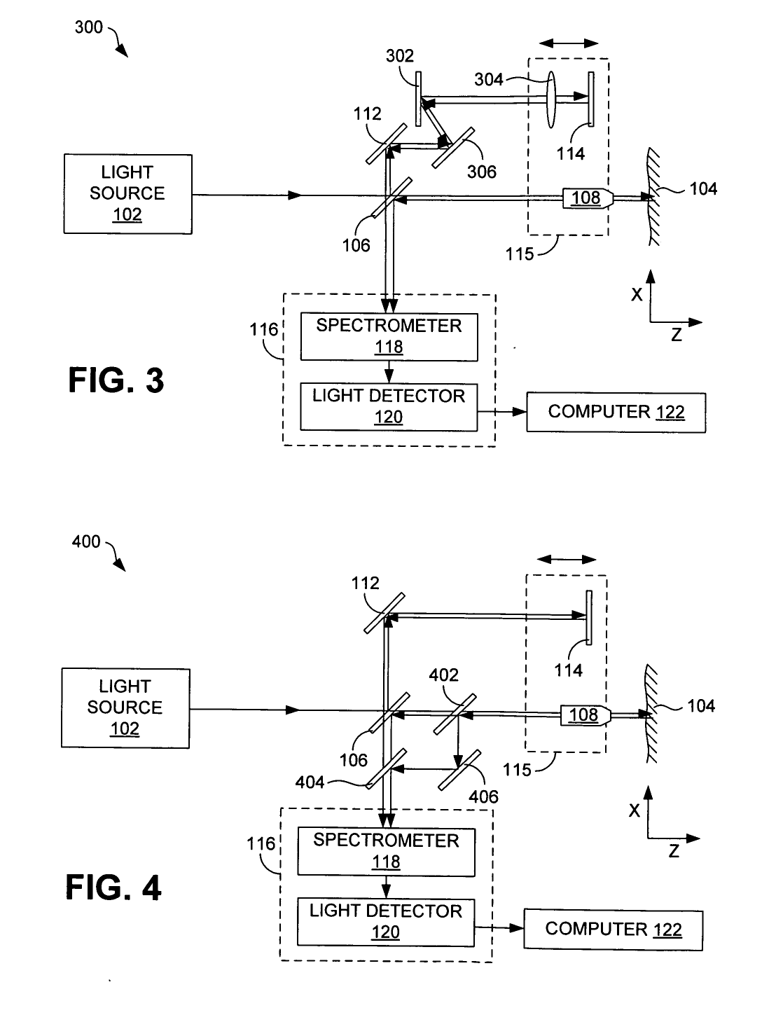

FIG. 3 illustrates a second embodiment of a system 300 for simultaneously performing OCT and fluorescence spectroscopy. The system 300 is similar to the system 100 of FIG. 1 and therefore comprises several of the components of the system 100, which perform the same functions. In addition, however, the system 300 further includes a reflective grating 302 and a lens 304 that form a Fourier-domain optical delay line that compensates for dispersion mismatch between the sample path and the reference path of the system by automatically adjusting the distance between the grating and the focal plane of the lens along the depth of imaging. As indicated in FIG. 3, light emitted by the light source 102 is directed toward the grating 302 with a mirror 306.

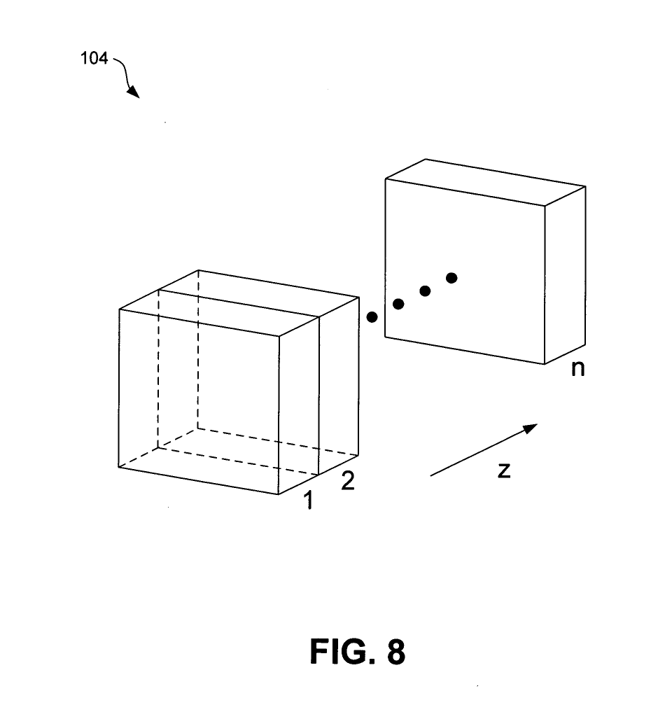

Referring to FIG. 8, depicted is imaging various target portions or zones of the material 104 under evaluation that explains use of the optical delay line. A target zone 1 can be imaged and analyzed by scanning the material laterally in a plane perpendicular to the optical axis of the objective 108 (FIG. 1). A target zone 2, which is deeper than target zone 1, can be focused by shortening the distance between the objective 108 and the material 104. Dispersion mismatch between the reference and sample paths caused by the deeper imaging position of the target zone 2 can, for example, be compensated by automatically increasing the distance between the grating 302 and the lens 304 of the optical delay line, which is adapted for dispersion compensation.

FIG. 4 illustrates a third embodiment of a system 400 for simultaneously performing OCT and fluorescence spectroscopy. The system 400 is similar to the system 100 of FIG. 1 and therefore comprises several of the components of the system 100, which perform the same functions. In addition, however, the system 400 further includes cold mirrors 402 and 404. The cold mirrors 402, 404 are configured to transmit NIR light and reflect fluorescent light. With such an arrangement, all of the fluorescent light emitted by the material 104 under evaluation is delivered to the receiver 116, instead of a portion of that light being transmitted through the beam splitter 106. In such an arrangement, the beam splitter 106 can operate only in the NIR region. Accordingly, less of the fluorescent signal is lost with the system 400, and the system is more efficient from a fluorescence spectroscopy perspective. As indicated in FIG. 4, light reflected by the first cold mirror 402 is reflected toward the second cold mirror 404 with a conventional mirror 406.

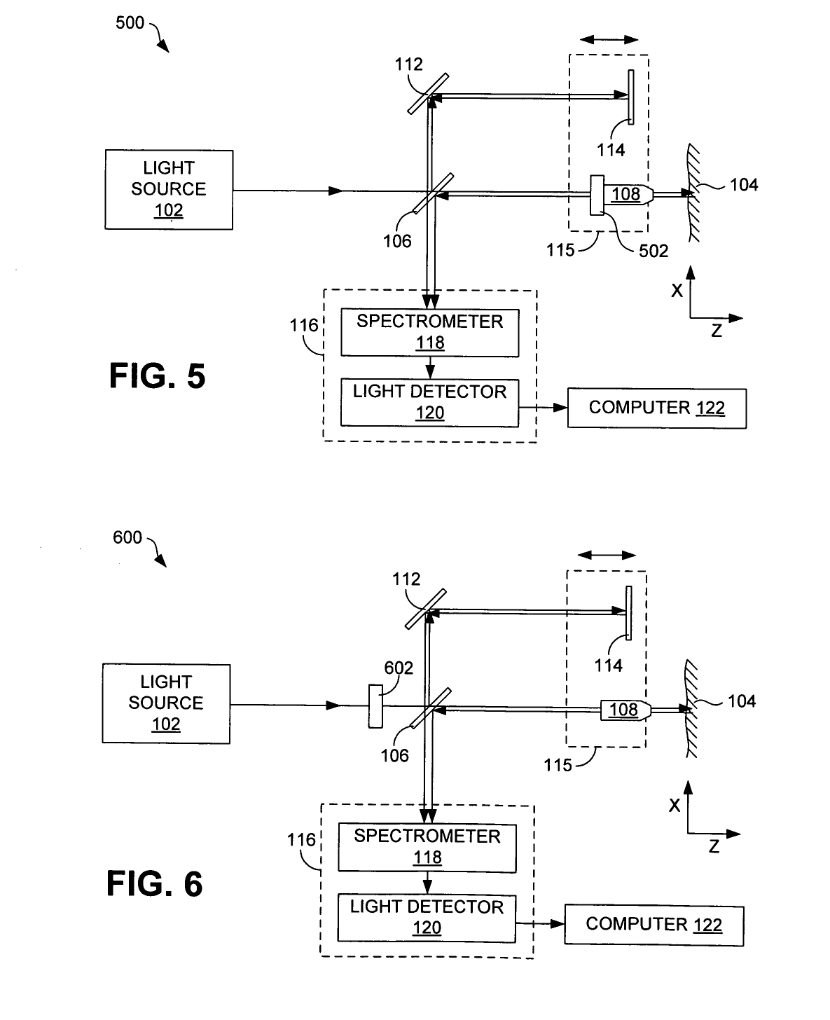

FIG. 5 illustrates a fourth embodiment of a system 500 for simultaneously performing OCT and fluorescence spectroscopy. The system 500 is similar to the system 100 of FIG. 1 and therefore comprises several of the components of the system 100, which perform the same functions. In addition, however, the system 500 further includes a dispersion compensator 502 that compensates for chromatic dispersion that can occur in the pulsed light signals from the light source 102 as they are transmitted through the system to avoid broadening of pulses from the light source, which lowers the peak power of the pulses. In the embodiment of FIG. 5, the compensator 502 is positioned adjacent to or within the objective 108.

FIG. 6 illustrates a fifth embodiment of a system 600 for simultaneously performing OCT and fluorescence spectroscopy. The system 600 is similar to the system 100 of FIG. 1 and therefore comprises several of the components of the system 100, which perform the same functions. In addition, however, the system 600 further includes a dispersion compensator 602 that, like dispersion compensator 502, compensates for chromatic dispersion. The compensator 602 is positioned adjacent the light source 102.

FIG. 7 illustrates a sixth embodiment of a system 700 for simultaneously performing OCT and fluorescence spectroscopy. The system 700 is similar to the system 100 of FIG. 1 and therefore comprises several of the components of the system 100, which perform the same functions. In addition, however, the system 700 includes a reflective grating 702 and a lens 304 that form a Fourier-domain optical delay line that compensates for dispersion mismatch. Moreover, the system 700 includes a scanning mirror 706 positioned between the light source 102 and the objective 108. The scanning mirror 706 is pivotally adjustable, as indicated by arrows 708. In use, the scanning mirror 706 can be pivoted to modify the angle at which light from the light source 102 reaches the objective 108. Through that modification, the material 104 under evaluation can be scanned in the x and/or y direction(s).

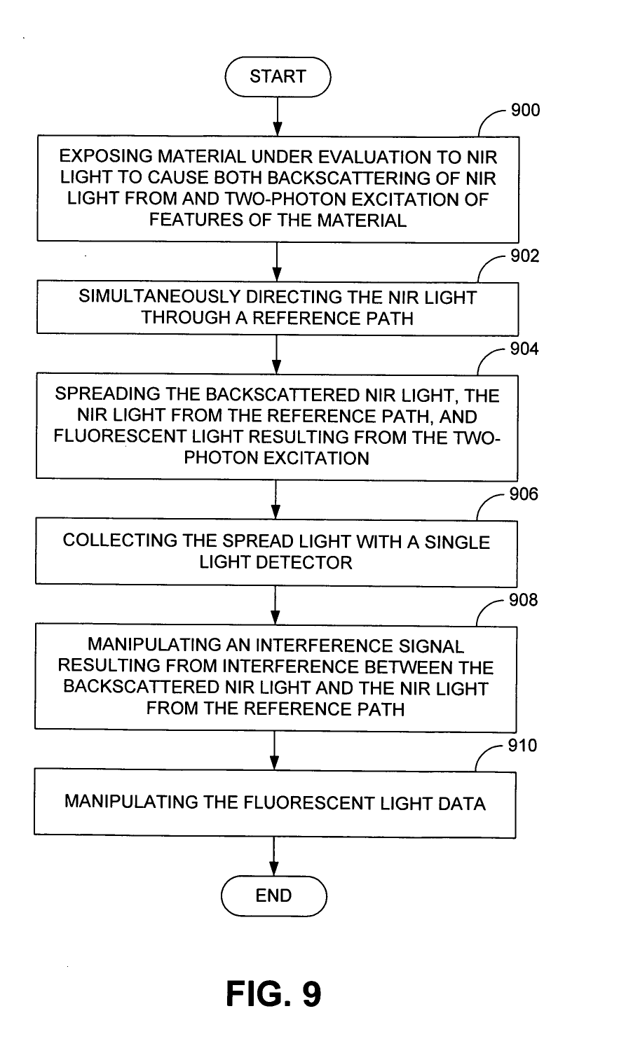

In accordance with the above disclosure, a method for simultaneously performing OCT and fluorescence spectroscopy can be described as that illustrated in flow diagram of FIG. 9. Beginning with block 900, material under evaluation is exposed to NIR light to cause both backscattering of NIR light from and two-photon excitation of features of the material. Simultaneously, NIR light is directed through a reference path, as indicated in block 902.

The method further comprises spreading the backscattered NIR light, the NIR light from the reference path, and the fluorescent light resulting from the two-photon excitation, as indicated in block 904. Next, the spread light is collected with a single light detector, as indicated in block 906. With reference to block 908, an interference signal resulting from the interference between the backscattered NIR light and the NIR light from the reference path is manipulated. By way of example, frequency-domain analysis, for instance Fourier-transform analysis, can be performed to generate an OCT image. In addition, as indicated in block 910, the fluorescent light data is manipulated, for example to generate a fluorescence spectroscopy image.

As stated above, while particular embodiments have been described in this disclosure, alternative embodiments are possible. For example, although various embodiments have been described that comprise discrete components, it is to be understood that further alternative embodiments may comprise hybrid embodiments that include one or more components of the alternative embodiments. For instance, one such hybrid embodiment may comprise one or more of the grating and lens of FIG. 3, the cold mirrors of FIG. 4, a dispersion compensator as indicated in FIGS. 5 and 6, and the scanning mirror of FIG. 7. In other words, the disclosed embodiments are not necessarily mutually exclusive.

1. An imaging system, comprising
a light source that emits light to be delivered to a material under evaluation; and
a receiver that collects both light that is backscattered by features of the material and fluorescent light that is emitted by features of the material, such that separate apparatuses are not needed to collect both the backscattered light and the fluorescent light.

2. The system of claim 1, wherein the light source is a low-coherence, near-infrared light source.

3. The system of claim 2, wherein the light source emits light having a central wavelength within the range of approximately 700 nanometers to 900 nanometers.

4. The system of claim 2, wherein the light source emits light having a central wavelength of approximately 800 nanometers.

5. The system of claim 1, wherein light emitted by the light source causes both the backscattering of light and generation of the fluorescent light, the backscattered light being in the near-infrared spectrum and the fluorescent light being in the visible spectrum.

6. The system of claim 1, wherein the receiver comprises a spectrometer that spreads the received light by wavelength and a single light detector that receives the spread light.

7. The system of claim 6, wherein the light detector comprises one of a charge-coupled device, photodiode array, or a photomultiplier array.

8. An imaging system for simultaneously performing Fourier-domain optical coherence tomography (OCT) and two-photon fluorescence spectroscopy on a material under evaluation, the system comprising:
a low-coherence, near-infrared light source that emits high-power, near-infrared light that causes both backscattering of near-infrared light from features in the material and two-photon excitation of features in the material, the two-photon excitation generating fluorescent light; and
a receiver comprising a single light detector that collects both the backscattered near-infrared light and the fluorescent light so as to enable both Fourier-domain OCT and fluorescence spectroscopy.

9. The system of claim 8, wherein the light source emits light having a central wavelength of approximately 800 nanometers such that the backscattered near-infrared has a central wavelength of approximately 800 nanometers and the fluorescent light emits in the near-infrared and visible spectrum with wavelengths ranging from approximately 350 nanometers to 700 nanometers.

10. The system of claim 8, wherein the light source is a titanium-doped sapphire laser.

11. The system of claim 8, wherein the receiver further comprises a spectrometer that spreads received light across the light detector by wavelength such that the backscattered near-infrared light is received by a portion of the light detector that is different from a portion of the light detector that receives the fluorescent light.

12. The system of claim 8, wherein the light detector comprises one of a charge-coupled device, photodiode array, or a photomultiplier array.

13. The system of claim 8, further comprising a sample path that transmits light emitted by the light source to the material and that transmits the backscattered near-infrared light and the fluorescent light to the receiver.

14. The system of claim 13, further comprising a reference path that transmits light emitted by the light source to a reference mirror and then to the receiver for the purpose of generating an interference signal resulting from combination of the backscattered near-infrared light and the near-infrared light emitted by the light source.

15. The system of claim 8, further comprising a Fourier-domain optical delay line that compensates for dispersion mismatch.

16. The system of claim 8, further comprising at least one cold mirror that transmits near-infrared light and reflects fluorescent light.

17. The system of claim 8, further comprising a dispersion compensator that compensates for chromatic dispersion.

18. The system of claim 8, further comprising a scanning mirror that modifies an angle at which light from the light source reaches an objective to enable scanning of the material under evaluation.

19. A method for performing simultaneous optical coherence tomography (OCT) and fluorescence spectroscopy, the method comprising:
exposing a material under evaluation to near-infrared light to cause both backscattering of near-infrared light from and two-photon excitation of features of the material, the two-photon excitation resulting in generation of fluorescent light;
collecting the backscattered near-infrared light and the fluorescent light with a single light detector; and
manipulating data output by the light detector.

20. The method of claim 19, simultaneous to exposing the material under evaluation, directing reference near-infrared light through a reference path and collecting the reference near-infrared light at the light detector such that the near-infrared light and the backscattered reference near-infrared light interferes with each other.

21. The method of claim 19, further comprising spreading the backscattered near-infrared light, the reference near-infrared light, and the fluorescent light by wavelength prior to collection by the light detector such that near-infrared light and fluorescent light are received by different portions of the light detector.

22. A method for evaluating a material under consideration, the method comprising:
simultaneously collecting near-infrared light backscattered by the material and fluorescent light emitted by the material using a single light detector.
